(19) United States
(12) Patent Application Publication
(10) Pub. No.: US 2022/0404367 A1
(43) Pub. Date: Dec. 22, 2022

(54) NOVEL ANTIBODIES FOR DETECTING GASTRIC CANCER

(71) Applicants: Joshua LaBaer, Chandler, AZ (US); Ji Qiu, Chandler, AZ (US); Lusheng Song, Tempe, AZ (US); Yunro Chung, Chandler, AZ (US)

(72) Inventors: Joshua LaBaer, Chandler, AZ (US); Ji Qiu, Chandler, AZ (US); Lusheng Song, Tempe, AZ (US); Yunro Chung, Chandler, AZ (US)

(21) Appl. No.: 17/761,653

(22) PCT Filed: Sep. 24, 2020
(86) PCT No.: PCT/US20/52561

§ 371 Date: Mar. 18, 2022

Related U.S. Application Data
(60) Provisional application No. 62/904,833, filed on Sep. 24, 2019.

(51) Int. Cl. G01N 33/574; C07K 16/12; G01N 33/543; G01N 33/569

(52) U.S. Cl. CPC G01N 33/57446; C07K 16/121; G01N 33/543; G01N 33/56922; G01N 2333/205; G01N 2469/20; G01N 2800/7028

(57) ABSTRACT

Provided herein are methods, compositions, kits, and systems for diagnosing, predicting, and treating gastric cancer for a subject based on the presence and level of antibodies against particular H. pylori proteins in a biological sample obtained from the subject. In particular, provided herein are methods for identifying a subject having increased risk of developing gastric cancer, methods for detecting gastric cancer in a subject, methods for determining an H. pylori antibody signature comprising antibodies, contained in a biological sample from a subject, that specifically bind to immobilized H. pylori antigens, and kits comprising components and instructions for performing the methods of this disclosure.

### CROSS-REFERENCE TO RELATED APPLICATIONS

This application claims priority to U.S. Provisional Application No. 62/904,833, filed Sep. 24, 2019, which is hereby incorporated by reference herein in its entirety.

### STATEMENT REGARDING FEDERALLY SPONSORED RESEARCH

This invention was made with government support under R01 CA199948 awarded by the National Institutes of Health. The government has certain rights in the invention.

### BACKGROUND

Gastric cancer (GC) is a major public health burden, representing the third leading cause of cancer mortality in the world with ˜780,000 deaths in 2018. Noncardiacc GC, an anatomical subsite that excludes the most proximal portion of the stomach (cardiac GC), contributes to more than 80% of newly diagnosed GC cases. Helicobacter pylori (H. pylori) chronic infection is the primary causative factor attributed to ˜90% of noncardiacc GC. Although H. pylori infection is common, progression to GC is a rare consequence. The reasons why only a subset of infected individuals develop GC have not been fully elucidated.

Current knowledge of chronic H. Pylori infection is mostly based on detection of antibodies to either whole-cell antigen preparations or the cytotoxin-associated gene A (CagA) virulence factor. These antibodies have been consistently associated with GC risk. Using two-dimensional gel electrophoresis and liquid chromatography-mass spectrometry, a few immunogenic proteins of H. pylori have been identified and used to develop multiplex sero-assays. These more specific serologic methods have identified modest but variable associations with GC risk for antibodies to several more bacterial proteins, including GroEL, HcpC, Omp and HP0305, but, to date, none of the proposed markers has sufficient discriminatory power to distinguish GC patients from cancer-free individuals. Many of the relevant studies obtained samples with matched demographic factors (e.g., age, sex, race, etc.) between cancer cases and controls but without considering background differences in H. Pylori infection, such as prevalence rates and bacterial strains. Thus, inconsistency of biomarker performance between studies might be due to population differences in infection rates or strain differences in virulence. Furthermore, conventional techniques such as gel electrophoresis-based methods lacked sufficient resolution or detection limits to profile reactivity to the entire set of immunogenic proteins. Accordingly, there remains a need in the art for improved reagents and methods for detecting gastric cancer, assessing risk of developing gastric cancer, and identifying subjects in need of treatment for gastric cancer, meaning reagents and methods that are more reliable and have sufficient discriminatory power for risk stratification and for early detection in asymptomatic individuals.

### SUMMARY

In a first aspect, provided herein is a method for identifying a subject having increased risk of developing gastric cancer. The method can comprise or consist essentially of (a) reacting a biological sample obtained from a subject with a reagent composition that comprises components for detecting in the biological sample the presence of one or more antibodies selected from anti-HP1118/Ggt, anti-HP0516/HslU, anti-HP0243/NapA, anti-HP1293/RpoA, anti-HP0371/FabE, and anti-HP0875/KatA; and (b) detecting the presence of the antibodies in the sample, wherein reduced seroreactivity relative to a control for one or more of the antibodies is indicative of a 2- to 8-fold increased risk of gastric cancer. The detected antibodies can comprise anti-HP1118, anti-HP0516, and anti-HP0243. The detected antibodies can comprise anti-HP1118/Ggt, anti-HP0516/HslU, anti-HP0243/NapA, anti-HP1293/RpoA, anti-HP0371/FabE, and anti-HP0875/KatA. The reagent composition can further comprise a component for detecting the presence and level of anti-CagA antibodies in the sample, and wherein increased seroreactivity for anti-CagA antibodies can be indicative of an increased risk of gastric cancer relative to a control. The detected antibodies can comprise anti-HP1118/Ggt, anti-HP0516/HslU, anti-HP0243/NapA, and anti-CagA. The detected antibodies can comprise anti-HP1118/Ggt, anti-HP0516/HslU, anti-HP0243/NapA, anti-HP1293/RpoA, anti-HP0371/FabE, anti-HP0875/KatA, and anti-CagA. The method can further comprise identifying the subject has having an increased risk of developing a diffuse-type gastric cancer tumor if the subject has a higher anti-HP1118/Ggt response relative to an intestinal-type cancer control. The method can further comprise identifying the subject has having an increased risk of developing a noncardiac-type gastric cancer tumor if the subject has a higher anti-HP1118/Ggt response relative to a cardiac-type gastric cancer control. The method can further comprise administering a vaccine-based gastric cancer treatment to the subject if identified as having an increased risk of gastric cancer. The biological sample can be one or more of a whole blood sample, a serum sample, and a plasma sample. The method can detect gastric cancer prior to symptom onset.

In another aspect, provided herein is a method to detect gastric cancer. The method can comprise or consist essentially of (a) reacting a biological sample obtained from a subject with a reagent composition that comprises components for determining a level of antibodies to one or more of 53 H. pylori proteins listed in Table 1 are present in the sample; (b) determining levels of the antibodies in the biological sample; and (c) comparing the levels to predetermined values indicative of gastric cancer, wherein if the level of antibodies in the biological sample falls within the predetermined values indicative of gastric cancer, the level in the biological sample indicates that the subject has gastric cancer. The antibodies can comprise anti-HP1118/Ggt, anti-HP0516/HslU, anti-HP0243/NapA. The detected antibodies can comprise anti-HP1118/Ggt, anti-HP0516/HslU, anti-HP0243/NapA, anti-HP1293/RpoA, anti-HP0371/FabE, and anti-HP0875/KatA. The reagent composition can further comprise a component for determining a level of anti-CagA antibodies in the sample, wherein if the level of anti-CagA antibodies in the biological sample falls within anti-CagA antibody levels of a reference sample obtained from an individual having gastric cancer, the level in the biological sample indicates that the subject has gastric cancer. The predetermined values can be obtained from a reference sample obtained from an individual or a group of individuals having gastric cancer. In some cases, the method further comprises administering a gastric cancer treatment to the subject if identified as having gastric cancer. The biological sample can be one or more of a whole blood sample, a serum sample, and a plasma sample. The determining step can be carried out using an ELISA assay or a Western Blot assay.

In a further aspect, provided herein is a method of determining an H. pylori antibody signature comprising antibodies, contained in a biological sample from an individual, that specifically bind to immobilized H. pylori antigens. The method can comprise or consist essentially of (a) contacting the sample to a panel of immobilized H. pylori antigens under conditions that promote formation of antigen-antibody complexes; and (b) identifying complexes formed by immobilized H. pylori antigens and antibody in the sample, to determine an H. pylori antibody signature. The antibody signature can be a level of antibody specifically binding to each immobilized antigen. In some cases, the method further comprises comparing an antibody signature from one individual to the antibody signature from another individual. One individual can have a disease process, and one individual can be a healthy individual, and the method can allow comparison of the antibody signature in the healthy individual and the individual with a disease. The disease process can comprise gastric cancer. The immobilized H. pylori antigens can comprise one or more of HP1118/Ggt, HP0516/HslU, HP0243/NapA, HP1293/RpoA, HP0371/FabE, and HP0875/KatA.

In another aspect, provided herein is a kit for determining and/or detecting at least one biomarker associated with gastric cancer, the kit comprising a reagent composition that comprises components for detecting in a biological sample the presence of one or more antibodies selected from anti-HP1118/Ggt, anti-HP0516/HslU, anti-HP0243/NapA, anti-HP1293/RpoA, anti-HP0371/FabE, and anti-HP0875/KatA.

In another aspect, provided herein is a kit for diagnosing a gastric cancer in a subject, the kit comprising a reagent composition that comprises components for detecting in a biological sample obtained from the subject the presence of one or more antibodies selected from anti-HP1118/Ggt, anti-HP0516/HslU, anti-HP0243/NapA, anti-HP1293/RpoA, anti-HP0371/FabE, and anti-HP0875/KatA.

### BRIEF DESCRIPTION OF THE DRAWINGS

The present invention will be better understood and features, aspects, and advantages other than those set forth above will become apparent when consideration is given to the following detailed description thereof. Such detailed description makes reference to the following drawings, wherein:

FIG. 1 presents an exemplary workflow for H. pylori immunoproteomic profiling.

FIGS. 2A-2B present heatmaps of antibodies with >10% seropositivity on H. pylori-NAPPA: (A) Top 53 IgG seropositive responses (B) Top 15 IgA seropositive responses.

FIGS. 3A-3B demonstrate distribution of ODs for the six ELISA validated antibodies distinguishing GC cases from controls in (A) discovery and (B) validation sets.

FIG. 4 is a graph demonstrating area under the curve (AUC) of the receiver operating characteristic curve analysis for control status based on the three top ELISA validated antibodies (Abs). AUC value for three Abs was 0.64, for anti-CagA was 0.59, and for all four Abs was 0.73.

FIG. 5 is a schematic illustrating a proposed model of increasing CagA seroreactivity in the gastric carcinogenesis cascade despite loss of other anti-H. pylori antibodies. NAG: non-atrophic gastritis, AG: Atrophic gastritis, IM: intestinal metaplasia, DYS: dysplasia.

FIGS. 6A-6B demonstrate quality control of H. pylori-NAPPA. Intra- and inter-array Pearson correlations of (A) anti-GST and (B) pooled sample.

FIGS. 7A-7I demonstrate GSEA of the 53 H. pylori proteins with >10% seroprevalence compared to 1474 proteins with <10% seroprevalence using (A) Chou-Fasman, (B) Karplus-Schulz and (C) Parker methods. Analyses of (D) protein length, (E) isoelectric, (F) aromaticity, (G) fraction of helix, fraction of (H) sheets and (I) turns.

FIGS. 8A-8B demonstrate distribution of anti-Ggt level by (A) anatomical location and (B) Lauren classification histology.

FIG. 9 demonstrates numbers of positive antibodies by H. pylori-NAPPA in GC cases and controls.

FIG. 10 demonstrates distribution of ODs of anti-Ggt, anti-NapA, and anti-RpoA level distributions by tumor stage.

While the present invention is susceptible to various modifications and alternative forms, exemplary embodiments thereof are shown by way of example in the drawings and are herein described in detail. It should be understood, however, that the description of exemplary embodiments is not intended to limit the invention to the particular forms disclosed, but on the contrary, the intention is to cover all modifications, equivalents and alternatives falling within the spirit and scope of the invention as defined by the appended claims.

### DETAILED DESCRIPTION OF THE DISCLOSURE

All publications, including but not limited to patents and patent applications, cited in this specification are herein incorporated by reference as though set forth in their entirety in the present application.

The methods, devices, combinations, kits, and systems for diagnosing, predicting the risk of, and treating gastric cancer provided herein are based at least in part on the inventors' development and validation of a panel of protein biomarkers useful for distinguishing gastric cancer patients from healthy controls in discovery samples and validation samples. In particular, this disclosure relates to the development and validation of unique Helicobacter pylori (H. pylori) immunoproteomic profiles useful for identifying subjects having an increased risk of gastric cancer relative to subject-matched controls. An interesting and unexpected finding of this investigation was that Ig levels to particular H. pylori proteins negatively correlate with risk of gastric cancer.

Chronic H. pylori infection is the major risk factor for gastric cancer (GC). However, only some infected individuals develop GC after an H. pylori infection. As described herein, methods and compositions have been developed to detect plasma antibodies to H. pylori as biomarkers for an increased risk of gastric cancer. In particular, the methods and compositions uniquely identify subjects having a decreased immune response to H. pylori proteins. Without being bound to any particular mechanism or mode of action, such subjects may have a reduced ability to mount an immune response against the H. pylori bacterium and, thus, are at greater risk for developing gastric cancer.

Accordingly, in a first aspect, this disclosure provides methods for identifying a subject as having increased risk of developing gastric cancer. In some cases, the method comprises (a) reacting a biological sample obtained from a subject with a reagent composition that comprises components for detecting in the serum sample the presence of antibodies specific to H. pylori proteins; and detecting the presence of the antibodies in the sample. As used herein, the term “gastric cancer” (also known as “stomach cancer”) refers to a cancer of the stomach or of stomach cells. Gastric cancer generally develops from neoplastic cells in the lining of the stomach (mucosa or stomach epithelium) and may be in pylorus, body, or cardiac (lower, body and upper) parts of the stomach. Gastric cancer often remains asymptomatic or exhibits only nonspecific symptoms in its early stages. Consequently, diagnosis in many cases is not made until the disease has reached an advanced stage. In some cases, the methods comprise measuring a level of a biomarker associated with gastric cancer in a biological sample obtained from a subject.

In preferred embodiments, the method comprises detecting and/or measuring a level of a biomarker such as, for example, an IgG or IgA antibody having specificity for H. pylori proteins. For example, target analytes include IgG specific antibodies targeting H. pylori proteins that show a statistically significant difference in gastric cancer diagnosis. In some cases, therefore, the methods comprise detecting IgG and IgA antibodies having specificity for H. pylori proteins in a sample obtained from a subject. As described and demonstrated in the Examples, reduced seroreactivity for antibodies specific to particular H. pylori proteins is associated with increased risk of gastric cancer relative to a reference sample (e.g., a sample obtained from a subject free of gastric cancer). The antibodies include those listed in Table 2. Preferably, the antibodies are anti-HP1118, anti-HP0516, anti-HP0243, anti-HP1293, anti-HP0371, and anti-HP0875 antibodies. Reduced seroreactivity for these antibodies, relative to the reference sample, is indicative of a 2- to 8-fold increased risk of gastric cancer. In various embodiments, diagnostic tests that use these biomarkers alone or in combination show a sensitivity and specificity of at least about 85%, at least about 90%, at least about 95%, at least about 98% and about 100%.

As used herein, the term “seroreactivity” refers to a level and/or presence of reactivity to specific antibodies in a sample (e.g., biological sample of a subject or a pooled sample from multiple subjects) as determined using with techniques known in the art, such as ELISA. As described in this disclosure, it was determined that reduced seroreactivity to particular antigen-specific antibodies relative to a control (e.g., a control sample obtained from a subject that does not have gastric cancer) is indicative of a 2- to 8-fold increased risk of gastric cancer. It will be understood that absolute seronegativity is not required to determine that a subject has increased risk of gastric cancer. Instead, reduced seroreactivity for IgG and IgA antibodies having specificity for H. pylori proteins in a sample obtained from a subject may indicate a 2- to 8-fold increased risk of gastric cancer. The decreased level of reactivity may be at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, at least or about a 95% decrease. In some cases, the control is a subject that is free of gastric cancer but who exhibits a similar rate of H. pylori infection (˜80%) to the test subject. As demonstrated herein, titers of the discovered and validated antibodies in samples obtained from gastric cancer patients were much lower than in samples obtained from persons free of gastric cancer but not necessarily seronegative.

As used herein, the terms “seronegativity” and “seronegative” refer to a reduced level or negative result (or a subject having a negative result) in a test of blood serum, e.g., obtaining a negative result for the presence of an antigen-specific antibody, relative to a control. The term seronegative can encompass patients for whom blood tests do not reveal the presence of particular antibodies, which can mean the patient does not possess the antibodies, or the patient possesses low levels of the antibodies that cannot be detected by a particular assay. As used herein, the terms “seropositivity” and “seropositive” refer to a positive result (or a subject having a positive result) in a test of blood serum, e.g., obtaining a positive result for the presence of an antigen-specific antibody. The term seropositive can encompass patients for whom blood tests reveal the presence of particular antibodies.

It will be advantageous in some cases to assay for the presence of anti-CagA antibodies in the biological sample. CagA is a well-studied virulence factor of H. pylori, and increased seroreactivity of serum for CagA has been associated with gastric cancer. Accordingly, anti-CagA antibody is considered to be a biomarker of gastric cancer. By including in the reagent composition a component for detecting the presence and level of anti-CagA antibodies in the sample, it is possible to assay for increased seroreactivity for anti-CagA antibodies, relative to a reference, to detect gastric cancer or an increased risk of gastric cancer. The increased level of reactivity may be at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, at least or about a 95% increase, at least or about at 100% increase, at least or about at 200% increase, or more.

In some cases, the method further comprises identifying a subject as having an increased risk of developing a diffuse-type gastric cancer tumor if the subject has a higher anti-HP1118 response relative to an intestinal-type cancer control.

Alternatively or additionally, the method can further comprise identifying a subject as having an increased risk of developing a noncardiac-type gastric cancer tumor if the subject has a higher anti-HP1118 response relative to a cardiac-type gastric cancer control.

In a further aspect, provided herein are methods for detecting gastric cancer in a subject. In some cases, the method comprises (a) reacting a biological sample obtained from a subject with a reagent composition that comprises components for determining a level of antibodies to one or more of 53 H. pylori proteins listed in Table 1 are present in the sample; (b) determining levels of the antibodies in the biological sample; and (c) comparing the levels to predetermined values indicative of gastric cancer, wherein if the level of antibodies in the biological sample falls within the predetermined values indicative of gastric cancer, the level in the biological sample indicates that the subject has gastric cancer. The predetermined values can be obtained from a reference sample obtained from an individual or a group of individuals (e.g., a cohort) having gastric cancer.

In preferred embodiments, the antibodies comprise one or more of anti-HP1118/Ggt, anti-HP0516/HslU, anti-HP0243/NapA, anti-HP1293/RpoA, anti-HP0371/FabE, and anti-HP0875/KatA. In some cases the antibodies are a panel comprises or consisting essentially of anti-HP1118/Ggt, anti-HP0516/HslU, anti-HP0243/NapA, anti-HP1293/RpoA, anti-HP0371/FabE, and anti-HP0875/KatA antibodies. In some cases the antibodies are a panel comprises or consisting essentially of anti-HP1118/Ggt, anti-HP0516/HslU, anti-HP0243/NapA antibodies.

In some cases, the method further comprises determining a level of anti-CagA antibodies in the biological sample obtained from the subject. In such cases, the reagent composition further comprises a component for determining a level of anti-CagA antibodies in the sample, wherein if the level of anti-CagA antibodies in the biological sample falls within anti-CagA antibody levels of a reference sample obtained from an individual having gastric cancer, the level in the biological sample indicates that the subject has gastric cancer.

In another aspect, provided herein are methods to determine risk of gastric cancer. In some cases, the method comprises (a) reacting a biological sample obtained from a subject with a reagent composition that comprises components for determining the level of antibodies to one or more H. pylori proteins present in the sample; (b) determining levels of the antibodies in the sample; and (c) comparing the levels of antibodies to predetermined values indicative of high risk of gastric cancer, wherein if the level of antibodies in the sample falls within the levels antibodies of a subject with high risk of gastric cancer, the level in the sample of the subject is predictive for the risk of gastric cancer in the subject. Preferably, the H. pylori proteins are selected from those listed in Table 1.

In some cases, the methods of the disclosure further comprise administering an effective amount of a treatment regimen to treat gastric cancer. In some cases, the treatment regimen comprises one or more of vaccine-based therapy, chemotherapy, hormonal therapy, radiotherapy, surgery, and immunotherapy. As demonstrated in the Examples, anti-CagA antibodies showed a positive association with GC in this study, whereas, inverse associations were found for anti-HP1118/Ggt, anti-HP0516/HslU, anti-HP0243/NapA, anti-HP1293/RpoA, anti-HP0371/FabE, and anti-HP0875/KatA. Accordingly, it will be advantageous in some cases for the treatment regimen comprise boosting production of anti-H. pylori antibodies such as anti-HP1118/Ggt, anti-HP0516/HslU, anti-HP0243/NapA, anti-HP1293/RpoA, anti-HP0371/FabE, and anti-HP0875/KatA in a subject identified as having gastric cancer or identified as having increased risk of gastric cancer. In such cases, the treatment regimen can comprise administering a vaccine-based gastric cancer treatment to the subject, whereby the vaccine boosts the subject's immune response to H. pylori proteins identified herein as diagnostic for gastric cancer.

As used herein, the term “individual,” which may be used interchangeably with the terms “patient” or “subject,” refers to one who receives medical care, attention or treatment and may encompass a human patient. As used herein, the term “individual” is meant to encompass a person who has a gastric cancer, is suspected of having gastric cancer, or is at risk of gastric cancer. As used herein, “at risk of gastric cancer” means that the subject may be asymptomatic or suffering from one or more symptoms of gastric cancer such as discomfort in the upper abdomen, a feeling of fullness, and the like, but has not been diagnosed with gastric cancer.

In preferred embodiments, the biological sample is a blood sample. Any suitable blood sample obtained from the subject may be used, including but not limited to whole blood, serum, and blood plasma. In a preferred embodiment, a blood plasma sample is used. Methods for obtaining and preparing blood samples are well known in the art; such methods include those described herein. In one embodiment, plasma is prepared by centrifuging a blood sample under conditions suitable for pelleting of the cellular component of the blood.

The methods for detecting gastric cancer of this disclosure can be used as methods for diagnosing gastric cancer, and are effective for detecting gastric cancer at an early stage and/or prior to symptom onset. As used herein, the term “symptom onset” refers to the time point where the subject presents one or more symptoms characteristic of gastric cancer. Exemplary symptoms of gastric cancer include but are not limited to stomach pain, fatigue, feeling bloated after eating, feeling full after eating small amounts of food, severe persistent heartburn, severe indigestion, unexplained persistent nausea, persistent vomiting, and unintentional weight loss. In another aspect, provided herein is a method for assessing the risk for gastric cancer in a subject, i.e., the likelihood of gastric cancer being present in the subject and/or the likelihood of the subject developing the disease at a later time.

As used herein, the terms “detect” and “detection” refer to identifying the presence, absence, or amount of the object to be detected. Standard detection methods include, for example, radioisotope immunoassay, an enzyme-linked immunosorbent assay (ELISA), SISCAPA (Stable Isotope Standards and Capture by Anti-Peptide Antibodies, mass spectrometry, immunofluorescence assays, Western blot, affinity chromatography (affinity ligand bound to a solid phase), fluorescent antibody assays, immunochromatography, and in situ detection with labeled antibodies. Although any appropriate method can be selected, taking various factors into consideration, ELISA methods are particularly sensitive.

The terms “biomolecular marker,” “biomarker,” or “marker” (also sometimes referred to herein as a “target analyte”) are used interchangeably and refer to a molecule whose measurement provides information as to the state of a subject. In various exemplary embodiments, the biomarker is used to assess a pathological state. Measurements of the biomarker may be used alone or combined with other data obtained regarding a subject in order to determine the state of the subject. In one embodiment, the biomarker is “differentially present” in a sample taken from a subject of one phenotypic status (e.g., having gastric) as compared with another phenotypic status (e.g., not having gastric cancer). In one embodiment, the biomarker is “differentially present” in a sample taken from a subject undergoing no therapy or one type of therapy as compared with another type of therapy. Alternatively, the biomarker may be “differentially present” even if there is no phenotypic difference, e.g. the biomarkers may allow the detection of asymptomatic risk. A biomarker may be determined to be “differentially present” in a variety of ways, for example, between different phenotypic statuses if the mean or median level (particularly the expression level of antibodies specific to the H. pylori proteins described herein) of the biomarker in the different groups is calculated to be statistically significant. Common tests for statistical significance include, among others, t-test, ANOVA, Kruskal-Wallis, Wilcoxon, Mann-Whitney and odds ratio.

The actual measurement of levels of a target analyte can be determined (for example, at the protein level) using any method(s) known in the art. A molecule or analyte such as a protein, polypeptide or peptide, or a group of two or more molecules or analytes such as two or more proteins, polypeptides or peptides, is “measured” in a sample when the presence or absence and/or quantity of said molecule or analyte or of said group of molecules or analytes is detected or determined in the sample, preferably substantially to the exclusion of other molecules and analytes. The terms “quantity,” “amount,” and “level” are synonymous and generally well-understood in the art. The terms as used herein may particularly refer to an absolute quantification of a molecule or an analyte in a sample, or to a relative quantification of a molecule or analyte in a sample, i.e., relative to another value such as relative to a reference value as taught herein, or to a range of values indicating a base-line expression of the molecule or analyte in a sample obtained from a healthy subject or, as appropriate, a sample obtained from a subject known to have gastric cancer and/or a particular type of gastric cancer. These values or ranges can be obtained from a single patient or from a group of patients.

A target analyte is differentially present between the two samples if the amount of the target analyte in one sample is statistically significantly different from the amount of the target analyte in the other sample. As used herein, the phrase “differentially expressed” refers to differences in the quantity and/or the frequency of a target analyte present in a sample taken from patients having, for example, a particular disease as compared to a control subject.

For example, without limitation, a target analyte can be a polypeptide that is present at an elevated level or at a decreased level in samples of patients having a particular condition as compared to samples of control subjects. A target analyte can be differentially present in terms of quantity, frequency or both. In some cases, a target analyte is differentially present between the two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

Alternatively (or additionally), a target analyte is differentially present between the two sets of samples if the frequency of detecting the target analyte in samples of patients suffering from a particular disease or condition is statistically significantly higher or lower than in the control samples. For example, a target analyte is differentially present between the two sets of samples if it is detected at least about 120%, at least about 130%, at least about 150%, at least about 180%, at least about 200%, at least about 300%, at least about 500%, at least about 700%, at least about 900%, or at least about 1000% more frequently or less frequently observed in one set of samples than the other set of samples.

Articles of Manufacture

In another aspect, provided herein is a kit for determining and/or detecting at least one biomarker associated with gastric cancer. In some cases, the kit comprises a reagent composition that comprises components for detecting in a biological sample the presence of one or more biomarkers of gastric cancer. In some cases, the biomarkers of gastric cancer are antibodies selected from anti-HP1118/Ggt, anti-HP0516/HslU, anti-HP0243/NapA, anti-HP1293/RpoA, anti-HP0371/FabE, and anti-HP0875/KatA. In some cases, the kit may further comprise instructions for detecting gastric cancer or identifying a subject has having increased risk of gastric cancer according to the methods provided herein. In some cases, the kit further comprises materials for obtaining and preserving a biological sample, for example, from an individual.

In another aspect, provided herein is a kit for diagnosing a gastric cancer in a subject. In some cases, the kit comprises a reagent composition that comprises components for detecting in a biological sample obtained from the subject the presence of one or more biomarkers diagnostic of gastric cancer. In some cases, the biomarkers diagnostic of gastric cancer are antibodies selected from anti-HP1118, anti-HP0516, anti-HP0243, anti-HP1293, anti-HP0371, and anti-HP0875. In some cases, the kit may further comprise instructions for detecting gastric cancer or identifying a subject has having increased risk of gastric cancer according to the methods provided herein. In some cases, the kit further comprises materials for obtaining and preserving a biological sample, for example, from an individual.

Unless defined otherwise, all technical and scientific terms used herein have the same meaning as commonly understood by one of ordinary skill in the art to which the invention pertains. All definitions, as defined and used herein, should be understood to control over dictionary definitions, definitions in documents incorporated by reference, and/or ordinary meanings of the defined terms.

The terms “comprising”, “comprises” and “comprised of” as used herein are synonymous with “including”, “includes” or “containing”, “contains”, and are inclusive or open-ended and do not exclude additional, non-recited members, elements, or method steps. The phraseology and terminology used herein is for the purpose of description and should not be regarded as limiting. The use of “including,” “comprising,” “having,” “containing,” “involving,” and variations thereof, is meant to encompass the items listed thereafter and additional items. Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein, the terms “approximately” or “about” in reference to a number are generally taken to include numbers that fall within a range of 5% in either direction (greater than or less than) the number unless otherwise stated or otherwise evident from the context (except where such number would exceed 100% of a possible value). Where ranges are stated, the endpoints are included within the range unless otherwise stated or otherwise evident from the context.

It should also be understood that, unless clearly indicated to the contrary, in any methods claimed herein that include more than one step or act, the order of the steps or acts of the method is not necessarily limited to the order in which the steps or acts of the method are recited.

The present invention has been described in terms of one or more preferred embodiments, and it should be appreciated that many equivalents, alternatives, variations, and modifications, aside from those expressly stated, are possible and within the scope of the invention. The invention will be more fully understood upon consideration of the following non-limiting Examples. The invention will be described in greater detail by way of specific examples. The following examples are offered for illustrative purposes, and are not intended to limit the invention in any manner.

### EXAMPLES

### Example 1—Helicobacter pylori Immunoproteomic Profiles in Gastric Cancer

Chronic Helicobacter pylori infection is the major risk factor for gastric cancer (GC). However, only some infected individuals develop this neoplasia. Previous serology studies have been limited by the small numbers of H. pylori antigens. This example describes development and validation of Nucleic Acid-Programmable Protein Array (NAPPA) for H. pylori, targeting both immunoglobulin G (IgG) and immunoglobulin A (IgA) antibodies. The arrays were applied to blood samples from gastric cancer (GC) patients and controls to evaluate associations with nearly the complete bacterial immunoproteome. Using a sample set with similar H. pylori infection prevalence in cancer cases and controls, immunodominant markers have been comprehensively characterized antibody-specific associations with GC were identified, with potential translational implications. This study represents the first comprehensive assessment of anti-H. pylori humoral profiles in GC. Using cases and controls with similarly high H. pylori infection prevalence, anti-CagA antibodies showed a positive association with GC in this study, whereas, inverse associations were found for anti-HP1118/Ggt, anti-HP0516/HslU, anti-HP0243/NapA, anti-HP1293/RpoA, anti-HP0371/FabE, and anti-HP0875/KatA. As described herein, decreased responses to multiple H. pylori proteins may reflect mucosal damage and decreased burden of bacterial proteins, while high levels of anti-H. pylori antibodies and/or their target proteins may protect against carcinogenesis.

Materials and Methods

Study population: Individuals included in this analysis were selected from a previous population-based GC case-control study in Poland. Two independent sample sets were randomly selected: a discovery and validation of 50 and 100 GC case-control pairs, respectively. Overall, the GC cases mainly represent advanced-stage disease (68%) and noncardiac tumors (76%). Cases and controls were comparable in distributions of age, sex, and similarly high prevalence antibodies to H. pylori (Table 1) using an enzyme-linked immunosorbent assay using whole H. pylori cell lysate (“wcELISA”).

The original study was approved by Institutional Review Boards in the Polish Sklodowska-Curie Memorial Cancer Centre and Institute of Oncology and the U.S. National Cancer Institute. Informed consent was obtained from all participants. The present case-control analysis was exempted by the U.S. National Institutes of Health (NIH) Office of Human Subjects Research Protection from institutional review board evaluation.

TABLE 1
H. pylori seropositivity by whole-cell ELISA (wcELISA) and selected characteristics
of gastric cancer patients and controls in discovery and validation sample sets

| | Discovery set | | Validation set | |
|---|---|---|---|---|
| | Gastric | Population- | Gastric | Population-based |
| | cancer cases | based controls | cancer cases | controls |
| | N = 50 | N = 50 | N = 100 | N = 100 |
| H. pylori seropositivity, n (%) | 40 (80) | 44 (88) | 76 (76) | 75 (75) |
| Age in years, median (range) | 65 (28-80) | 64 (28-79) | 65 (32-78) | 65 (33-79) |
| Male sex, n (%) | 34 (68) | 34 (68) | 64 (64) | 61 (61) |
| Advanced stage cancer*, n (%) | 35 (70) | — | 67 (67) | — |
| Noncardiac cancer, n (%) | 37 (74) | — | 77 (77) | — |
| Intestinal-type cancer, n (%) | 36 (72) | | 71 (71) | |

*Based on anti-H. pylori whole-cell ELISA;
**Advanced stage gastric cancer: Stage II to Stage IV.

Selection of H. pylori gene sequences: A total of 1527 H. pylori genes in Gateway Entry clones were obtained from the Biodefense and Emerging Infections (BEI) Research Resources Repository, U.S. National Institutes of Health, including 1453 clones from the reference strain 26695 (covering 91% of the full bacterial proteome) and 74 clones from strain J99 (12 genes with homology over 90% between these two reference strains). These clones were transferred into a NAPPA compatible pANT7-cGST expression vector using recombinational cloning. Sequences of CagA and vacA genes were not available in the BEI clone library, and hence not assessed by our H. pylori-NAPPA.

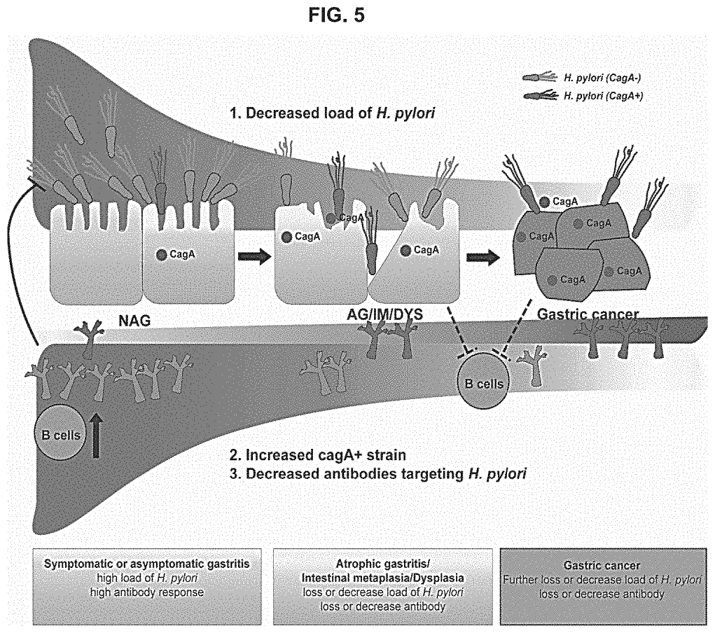

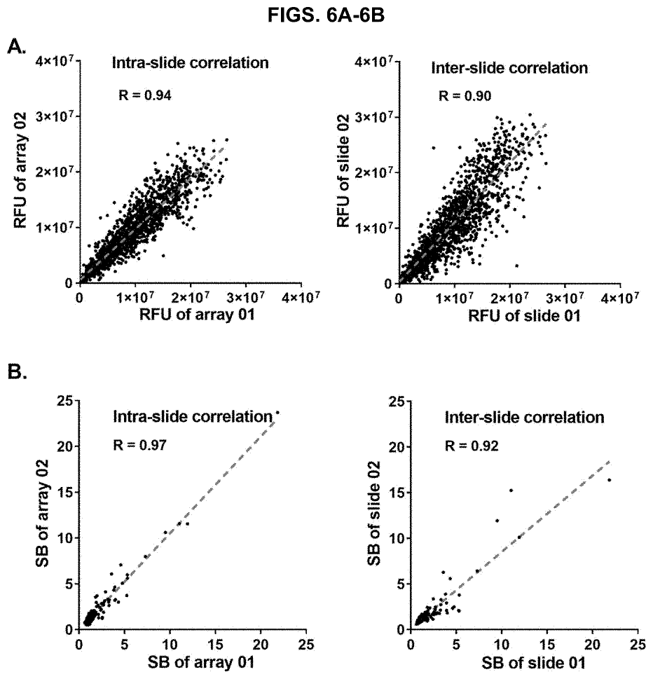

H. pylori NAPPA array fabrication, expression, and sample probing: H. pylori NAPPA arrays were fabricated in three main steps as previously described. Briefly, all DNA clones were printed sequentially into silicon nano-well substrates using piezoelectric dispensing system as array. At the time of usage, the NAPPA arrays were expressed by incubation with cell-free protein expression lysates at 30° C. for 2 hours and 15° C. for 30 minutes for protein expression and in situ capture, respectively. Reproducibility of H. pylori-NAPPA arrays was assessed using anti-GST protein displaying duplicates, for which intra-array and inter-array correlation coefficients were 0.94 and 0.90 (FIG. 5A), respectively. Isotype-specific (IgG and IgA) antibody profiling was performed by incubating the NAPPA arrays with 1:100 dilution of plasma followed by detection with Alexa 647 labeled goat anti-human IgG (H+L) and Cy3 labeled goat anti-human IgA. Arrays were scanned on a Tecan PowerScanner and raw fluorescence intensity data were extracted using ArrayPro Analyzer Software. Intra- and inter-slide correlations for an internal probing positive control and a pooled plasma combing all samples in the discovery set, were 0.97 and 0.92 respectively (FIG. 5B).

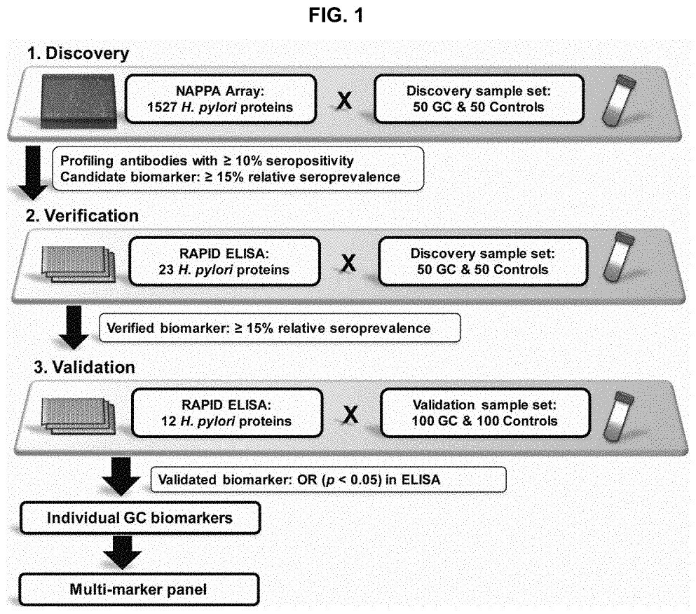

Antibody response on H. pylori-NAPPA was normalized as median normalized intensity (MNI) using the median of the raw signal intensities for all proteins on a given array.(14, 18) Based on our laboratory experience with NAPPA and ELISA assays, we interpreted MNI≥2.0 to indicate seropositive responses for a given bacterial antigen. Antibodies with >10% seropositivity in either GC cases or controls were selected and evaluated for their associations with GC. Relative seroprevalences for those antibodies were calculated as the percentage of cases or controls with MNI exceeding the 95^{th} percentile of the other group. Antibodies with >15% relative seroprevalence in either cases or controls were selected for ELISA verification using the same discovery sample set (FIG. 1).

Definition of immunodominant candidates, seropositivity and relative seroprevalence: We interpreted SB≥2.0 to indicate immunodominant responses for a given bacterial antigen. Immunodominant antibodies present in ≥10% of either cancer cases or controls were evaluated for their associations with GC. A seropositive antibody was defined as SB≥2.0 on NAPPA and was defined as OD 450 nm>0.1 on ELISA. Relative seroprevalence was calculated as the percentage of cases higher than the 95th percentile of controls or percentage of controls higher than the 95th percentile of cases.

Verification and validation using Rapid Antigenic Protein in situ Display (RAPID) ELISA: RAPID ELISA was performed as previously described. Briefly, 96-well plates (Corning, N.Y., USA) were first coated with goat anti-GST antibody (GE Healthcare Bio-Sciences, PA, USA) and incubated with an in vitro-expressed candidate antigen using cell-free protein expression lysates. After washing, 1:200 diluted samples were added followed by HRP-conjugated goat anti-human IgG (Jackson ImmunoResearch Labs, PA, USA). Plates were developed using TMB substrate (Thermo Fisher Scientific, MA, USA) and optical density at 450 nm (OD₄₅₀) was measured on a PerkinElmer Envision plate reader (Waltham, Mass., USA). In vitro expressed GST tag was used as a negative control. Antibody response was normalized by subtracting the OD₄₅₀ value of GST tag alone from the OD₄₅₀ of the GST-tagged target protein of the same sample. A seropositive antibody was defined as OD₄₅₀>0.1 and its relative seroprevalence was calculated accordingly.

Immunodominant antibodies selected from the NAPPA array were further calculated for relative prevalence. ELISA-verified antibodies with greater than 15% relative prevalence were further validated in an independent sample with ELISA. Sensitivity, specificity, and unadjusted odds ratios (ORs) were calculated. Antibodies with ORs<0.5 or >2 (p<0.05) were determined as passing validation (FIG. 1). We also tested for anti-CagA antibodies with ELISA using the commonly evaluated IVTT-expressed full-length CagA genes.

Association of antibodies with clinical parameters: Information on the following patient and tumor characteristics was available: age (≤55 vs.>55 years), alcohol consumption (never, current or former), smoking (never vs. ever), education (less than elementary school, completed elementary, higher than elementary), gender (male vs. female), family history of cancer (yes vs. no), stage of disease (localized, regional metastasis, distant metastasis, unspecified), histology (Lauren classification; intestinal, diffuse or unspecified), and tumor localization (cardiac, noncardiac, or overlapping). Significance of associations between patient characteristics and antibodies was determined by Wilcoxon-rank sum test.

Statistical analyses: Differences in the seropositive antibody numbers in GC cases and controls on H. pylori-NAPPA were assessed by the Wilcoxon rank-sum test. Kappa coefficients assessed agreement between available wcELISA results to the most immunogenic H. pylori-NAPPA antibody. Differences in the selected patient characteristics and anti-H. pylori antibodies were determined by Wilcoxon-rank sum test. Fisher's exact test was used to assess antigenic difference between potential antigenic membrane or secreted proteins (MSPs) compared to those at the other subcellular locations. Spearman's correlation coefficients were used to assess repeatability of response on H. pylori-NAPPA and on ELISA. ORs, 95% confidence intervals (95% CI) and corresponding p-values for ELISA validation were calculated based on Chi-square tests. Multi-marker models were built with Lasso logistic regressions with corresponding receiver operating characteristics (ROC) and area under the curve (AUC) values were calculated. We also performed leave-one-out cross validations. All statistical tests were two-sided and p-values<0.05 were considered statistically significant. All analyses were conducted with R version 3.6, Stata version 15 and GraphPad Prism 8.0.2.

In silico Functional Analysis: The Protein Subcellular Localization Prediction Tool (pSORT2) program was used to predict intracellular localization sites based on their sequences. Gene set enrichment analysis (GSEA) gseapy 0.9.3. was used to detect antigenic enrichment with three models (Chou-Fasman, Karplus Schulz, and Parker) as well as protein length, isoelectric point, aromaticity, and fraction of helix, sheets and turns.

Results

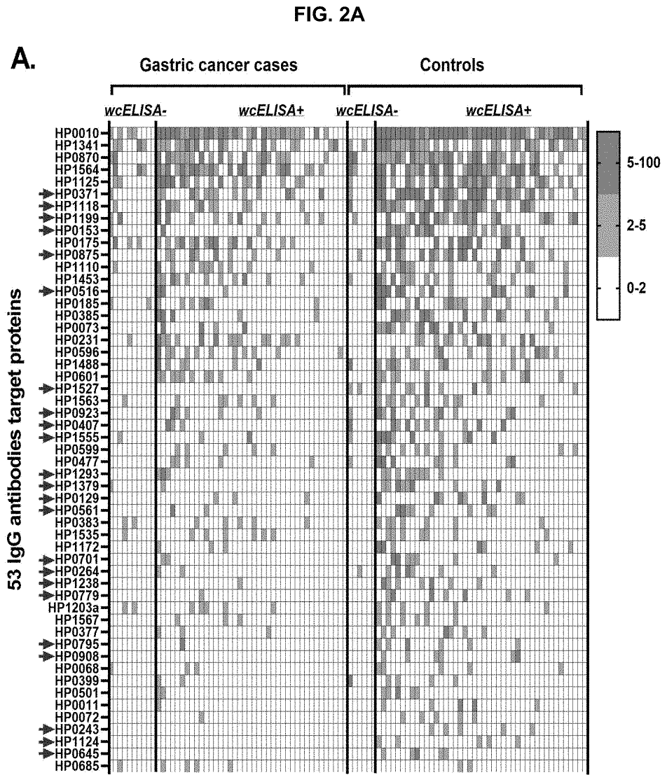

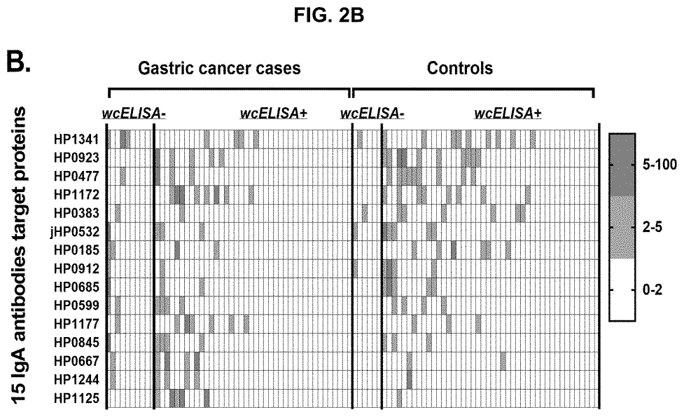

Anti-H. pylori antibody response on H. pylori-NAPPA: Out of 1527 proteins assayed on the H. pylori-NAPPA, 53 IgG and 15 IgA antibodies showed >10% seropositivity in the discovery sample set (Table 2, Table 4, FIG. 2). Overall, antibody response as measured by numbers of antibodies was significantly higher in controls (median, 16) as compared to GC cases (median, 10; p<0.01). The top five antibodies with the highest seropositivity in GC cases and controls combined were: Anti-HP0010/GroEL (77%), anti-HP1341/TonB (53%), anti-HP1564/PlpA (55%), anti-HP0870/FlgE (51%), and anti-HP1125/PalA (46%). All these antibodies had higher seropositivity in controls than in GC cases: anti-GroEL (84% controls vs. 70% cases), anti-TonB (62% vs. 44%), anti-FlgE (60% vs. 42%), anti-PlpA (58% vs. 52%), and anti-PalA (56% vs. 36%). The prevalence of anti-GroEL, the antibody with the highest seropositivity, aligned well with the high overall H. pylori seroprevalence as determined by wcELISA (kappa coefficient, 0.68).

Twenty-three anti-H. pylori antibodies with >15% relative seroprevalence in controls were selected for ELISA verification (Table 5). No antibodies showed >15% relative seroprevalence in GC cases. The IgA responses were low and were not selected for ELISA verification (Table 4).

TABLE 2
Seropositivity of 53 anti-H. pylori IgG antibodies on NAPPA

| | | | Seropositivity (%) | | |
|---|---|---|---|---|---|
| | | | All | | |
| Protein | Names | Full name | samples | Controls | Cases |
| HP0010 | GroEL, HSPb, | Molecular chaperone GroEL | 77 | 84 | 70 |
| | Hsp60, MopA | | | | |
| HP1341 | TonB2, TonB | Energy transducer TonB | 53 | 62 | 44 |
| HP0870 | FlgE | Flagellar hook protein FlgE | 51 | 60 | 42 |
| HP1564 | PlpA | ABC transporter substrate-binding protein | 55 | 58 | 52 |
| HP1125 | PalA | Peptidoglycan associated lipoprotein | 46 | 56 | 36 |
| HP0371 | AccB, FabE | Biotin carboxyl carrier protein | 42 | 56 | 28 |
| HP1118 | Ggt | Gamma-glutamyltranspeptidase | 36 | 48 | 24 |
| HP1199 | RpI2, Rpl7, RpIL | 50S ribosomal protein L7/L12 | 33 | 42 | 24 |
| HP0153 | RecA | Recombinase RecA | 19 | 32 | 6 |
| HP0175 | PpiC | Peptidylprolyl isomerase | 33 | 30 | 36 |
| HP0875 | KatA | Catalase | 23 | 30 | 16 |
| HP1110 | PorA | Pyruvate flavodoxin oxidoreductase subunit | 25 | 28 | 22 |
| | | alpha | | | |
| HP1453 | HomD | Membrane protein | 25 | 28 | 22 |
| HP0516 | HslU | ATP-dependent protease ATP-binding | 17 | 28 | 6 |
| | | subunit HslU | | | |
| HP0185 | | Hypothetical protein | 22 | 26 | 18 |
| HP0385 | | Hypothetical protein | 18 | 26 | 10 |
| HP0073 | UreA | Urease subunit alpha | 17 | 26 | 8 |
| HP0231 | | Protein-disulfide isomerase | 27 | 24 | 30 |
| HP0596 | | Tumor necrosis factor alpha-inducing | 23 | 24 | 22 |
| | | protein | | | |
| HP1488 | | Membrane protein | 18 | 24 | 12 |
| HP0601 | FlaA | Flagellin A | 21 | 22 | 20 |
| HP1527 | ComH | Hypothetical protein | 11 | 22 | 0 |
| HP1563 | TsaA | Peroxiredoxin | 17 | 20 | 14 |
| HP0923 | HopK, Omp22 | Membrane protein | 15 | 20 | 10 |
| HP0407 | BisC | Biotin sulfoxide reductase | 13 | 20 | 6 |
| HP1555 | Tfs | Elongation factor Ts | 13 | 20 | 6 |
| HP0599 | | Methyl-accepting chemotaxis protein | 14 | 18 | 10 |
| HP0477 | HopJ, Omp12 | Membrane protein | 14 | 18 | 10 |
| HP1293 | RpoA | DNA-directed RNA polymerase subunit | 12 | 18 | 6 |
| | | alpha | | | |
| HP1379 | Ion | ATP-dependent protease | 11 | 18 | 4 |
| HP0129 | | Hypothetical protein | 11 | 16 | 6 |
| HP0561 | FabG | 3-ketoacyl-ACP reductase | 10 | 16 | 4 |
| HP0383 | | Hypothetical protein | 14 | 14 | 14 |
| HP1535 | TnpA | Transposase | 14 | 14 | 14 |
| HP1172 | GlnH | Glutamine ABC transporter substrate- | 10 | 14 | 6 |
| | | binding protein GlnH | | | |
| HP0701 | GyrA | DNA gyrase subunit A | 9 | 14 | 4 |
| HP0264 | ClpB | Chaperone protein ClpB | 9 | 14 | 4 |
| HP1238 | AmiF | Formamidase | 8 | 14 | 2 |
| HP0779 | AcnB | Bifunctional aconitate hydratase 2/2- | 8 | 14 | 2 |
| | | methylisocitrate dehydratase | | | |
| HP1203a | SecE | Preprotein translocase subunit SecE | 13 | 12 | 14 |
| HP1567 | EngB | GTP-binding protein EngB | 9 | 12 | 6 |
| HP0377 | DsbC | Thiol:disulfide interchange protein DsbC | 9 | 12 | 6 |
| HP0795 | Tig | Trigger factor | 7 | 12 | 2 |
| HP0908 | FlgE | Flagellar hook protein FlgE | 6 | 12 | 0 |
| HP0068 | UreG | Urease accessory protein UreG | 8 | 10 | 6 |
| HP0399 | Rps1, RpsA | 30S ribosomal protein S1 | 7 | 10 | 4 |
| HP0501 | GyrB | DNA gyrase subunit B | 7 | 10 | 4 |
| HP0011 | GroES, HspA, | Co-chaperonin GroES | 6 | 10 | 2 |
| | Hsp10, MopB | | | | |
| HP0072 | UreB | Urease subunit beta | 6 | 10 | 2 |
| HP0243 | NapA | DNA protection during starvation protein | 5 | 10 | 0 |
| HP1124 | | Hypothetical protein | 5 | 10 | 0 |
| HP0645 | Slt | Lytic murein transglycosylase | 5 | 10 | 0 |
| HP0685 | FliP | Flagellar biosynthesis protein | 7 | 4 | 10 |

GSEA of target antigens of the most seroreactive anti-H. pylori antibodies: Out of the 1527 proteins, subcellular localizations of 908 were predicted using the pSORT2 program. Out of the 53 proteins targeted by these IgG antibodies with >10% seropositivity, 39 were predicted. Among these proteins, 11 (28%) were potential antigenic MSPs, indicating a >5-fold enrichment of MSPs overall (p<0.01). The MSPs elicited significantly higher rates of antibody response as compared to cytoplasmic proteins (p<0.01) (Table 6).

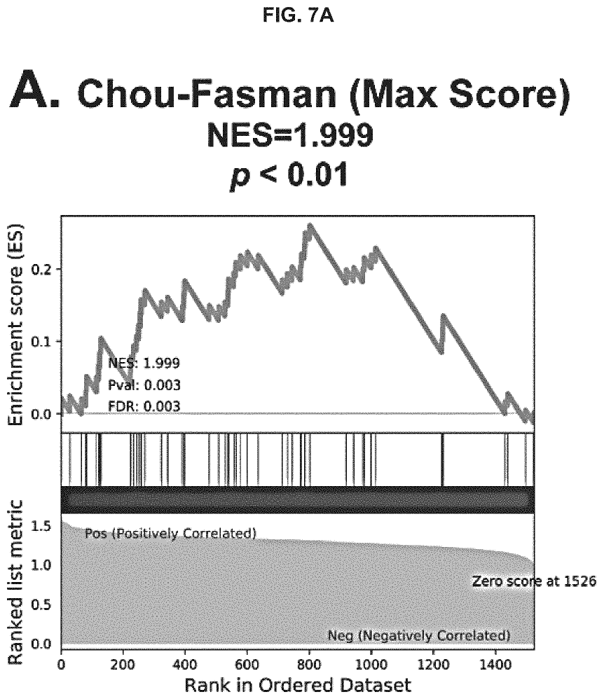

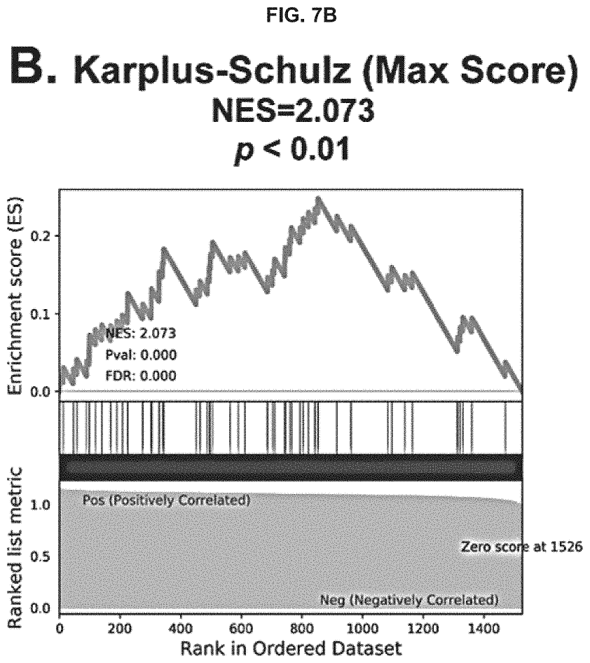

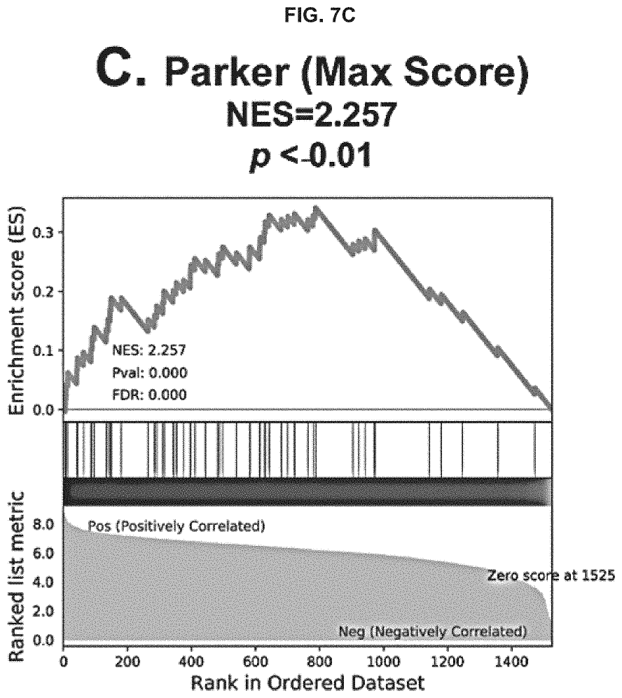

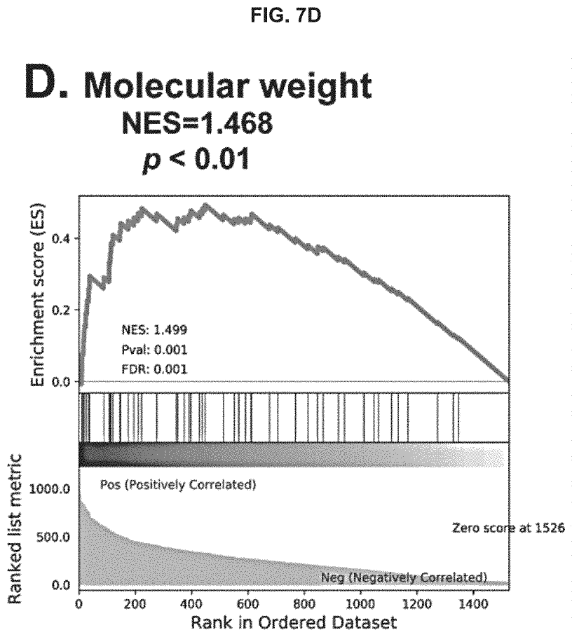

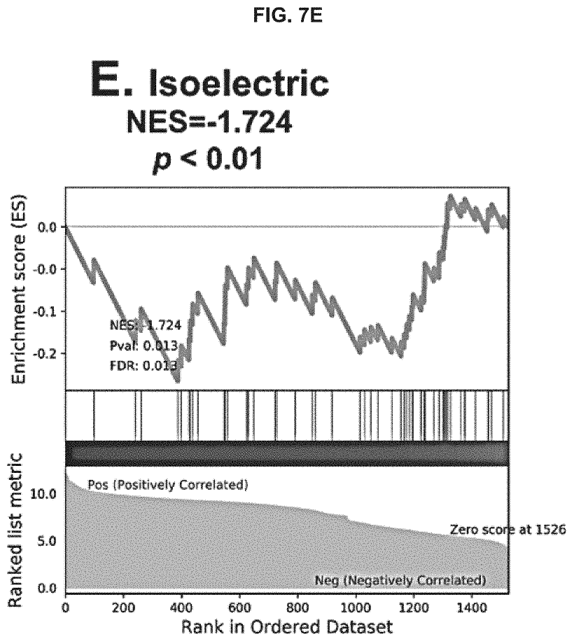

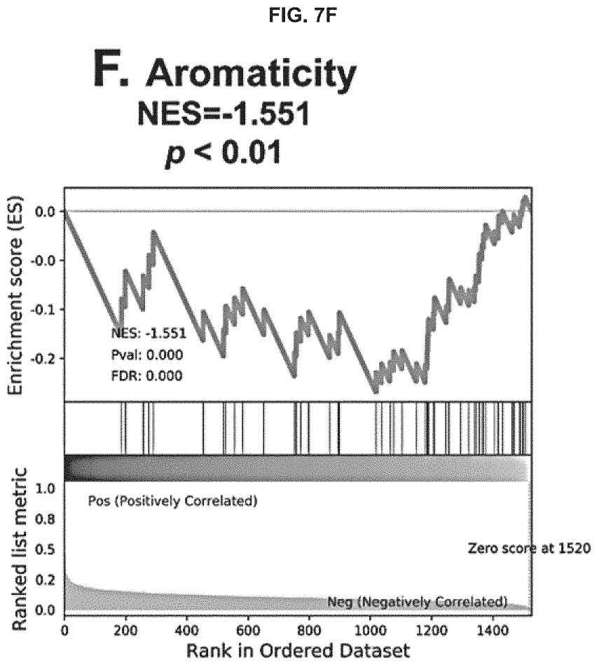

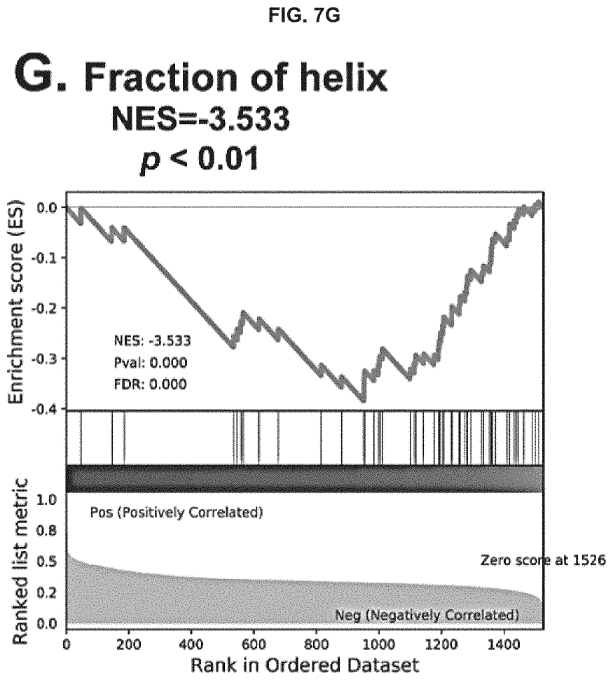

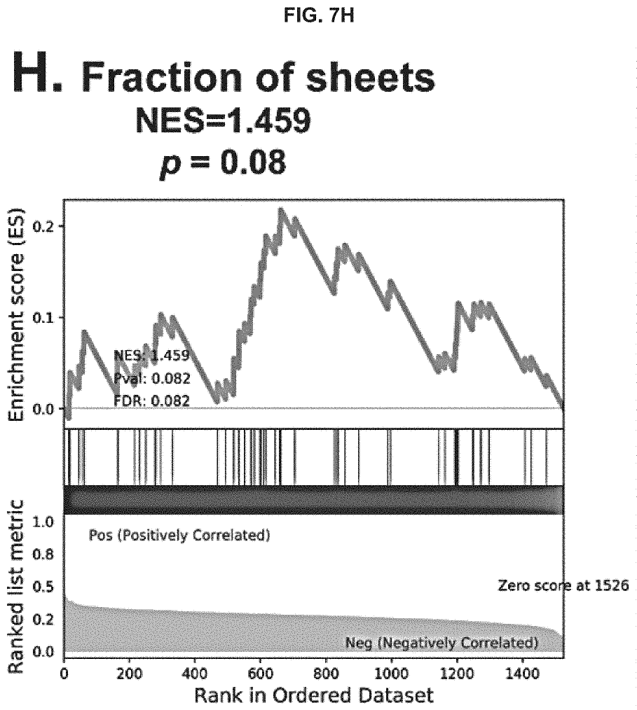

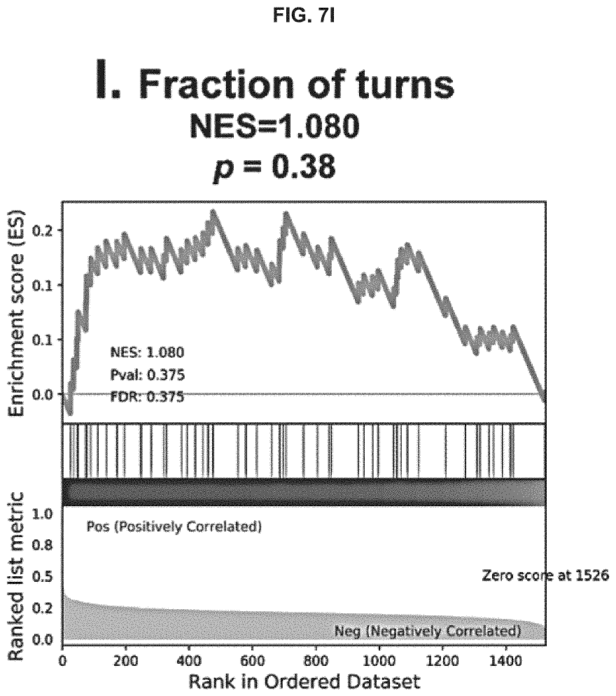

GSEA based on sequence-based biochemical characteristics and predicted antigenicity of the 53 antibody-targeted proteins showed a significant over-representation of proteins with high molecular weight, low isoelectric point, low aromaticity ratio, and low fraction of helix (FIGS. 7A-7I). The fractions of sheets and turns did not appear to contribute significantly to antigenicity.

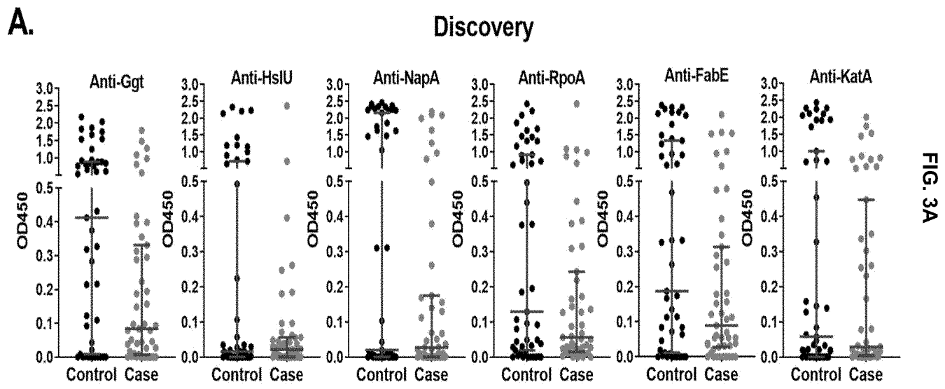

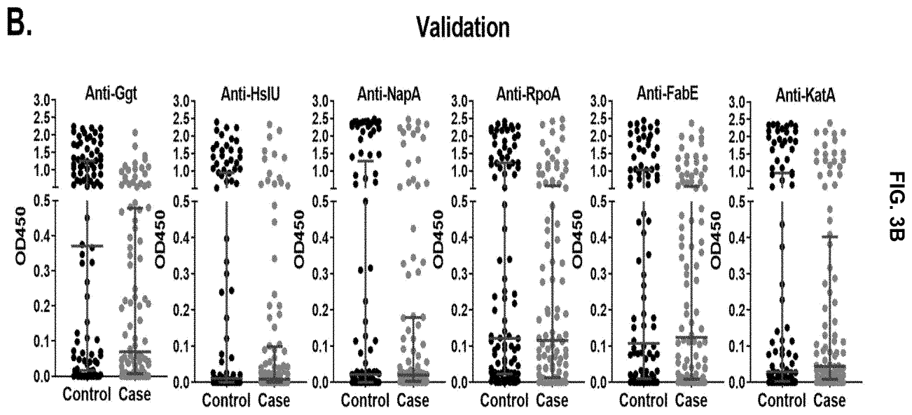

Verification and validation by RAPID ELISA: Using the same discovery sample set profiled on the H. pylori-NAPPA, we assayed the performance of the 23 candidate antibodies by ELISA. Spearman correlation coefficients between NAPPA and ELISA results had a median of 0.69 (25th and 75^{th} percentiles, 0.56 and 0.79). A total of 12 antibodies were ELISA verified with >15% relative seroprevalence in controls (Table 3). Six of the 12 antibodies were validated in an independent sample set: anti-HP1118/Ggt, anti-HP0516/HslU, anti-HP0243/NapA, anti-HP1293/RpoA, anti-HP0371/FabE, and anti-HP0875/FlgE (Table 3, FIG. 3).

TABLE 3
Relative seroprevalence among 23 candidate anti-H. pylori antibodies tested by ELISA

| | | Discovery | | | | | | |
|---|---|---|---|---|---|---|---|---|
| | | set | Validation set | | | | | |
| | | Percentage | Percentage | | | | | |
| | | in | in | Percentage | Odds | | | |
| Protein | Name | controls* | controls* | in cases* | Ratio | p-value | Validated | AUC |
| HP1118 | Ggt | 19 | 26 | 4 | 0.12 | 1.3E−05 | Yes | 0.63 |
| HP0516 | HslU | 30 | 32 | 16 | 0.40 | 8.1E−03 | Yes | 0.56 |
| HP0243 | NapA | 26 | 19 | 7 | 0.32 | 1.2E−02 | Yes | 0.51 |
| HP1293 | RpoA | 23 | 30 | 16 | 0.44 | 1.9E−02 | Yes | 0.55 |
| HP0371 | AccB, FabE | 19 | 18 | 7 | 0.34 | 1.9E−02 | Yes | 0.52 |
| HP0875 | KatA | 23 | 20 | 10 | 0.44 | 4.8E−02 | Yes | 0.51 |
| HP0795 | Tig | 17 | 23 | 13 | 0.50 | 6.6E−02 | No | |
| HP0153 | RecA | 30 | 23 | 15 | 0.59 | 1.5E−01 | No | |
| HP0701 | GyrA | 17 | 15 | 9 | 0.56 | 1.9E−01 | No | |
| HP0561 | FabG | 19 | 18 | 15 | 0.80 | 5.7E−01 | No | |
| HP0779 | AcnB | 28 | 14 | 16 | 1.17 | 6.9E−01 | No | |
| HP1379 | Ion | 19 | 18 | 17 | 0.93 | 8.5E−01 | No | |
| HP1527 | ComH | 13 | | | | | | |
| HP0407 | BisC | 13 | | | | | | |
| HP1555 | Tfs | 13 | | | | | | |
| HP0923 | HopK, Omp22 | 13 | | | | | | |
| HP1199 | RpI2, Rpl7, | 13 | | | | | | |
| | RpIL | | | | | | | |
| HP0129 | | 11 | | | | | | |
| HP0908 | FlgE | 9 | | | | | | |
| HP0645 | Sit | 9 | | | | | | |
| HP1124 | | 0 | | | | | | |
| HP0264 | ClpB | 0 | | | | | | |
| HP1238 | AmiF | 0 | | | | | | |

*Based on SB cutoff at 95^{th} percentile in cases (as determined in discovery set).

As a sensitivity analysis, we also compared the antibodies in all GC cases with H. pylori-positive controls by wcELISA. Nine antibodies, including all the six validated (as described above), had ORs with p<0.05 in the validation set (Table 7).

Anti-CagA was the only antibody showing higher seropositivity in GC cases compared to controls: 94% vs. 74% (OR, 5.5; 95% CI, 2.2-14.1), respectively.

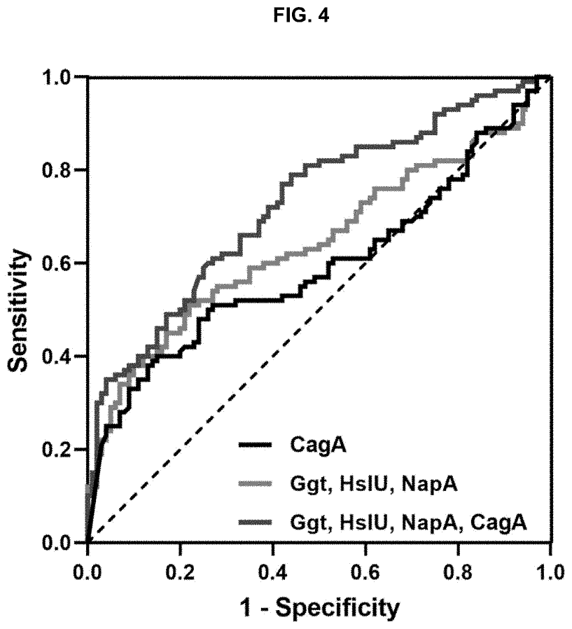

Discriminatory power of validated antibodies: All six ELISA validated antibodies each showed inverse associations with AUC values ranging between 0.51 and 0.63. Using Lasso regression, a three-antibody panel (anti-Ggt, anti-HslU, and anti-NapA) showed the highest AUC value of 0.64 (Table 3) among all combinations. When incorporating anti-CagA, AUC for the four-antibody panel was improved to 0.73 (FIG. 4).

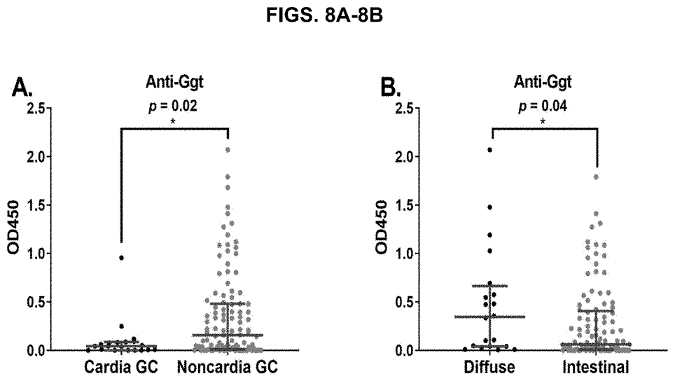

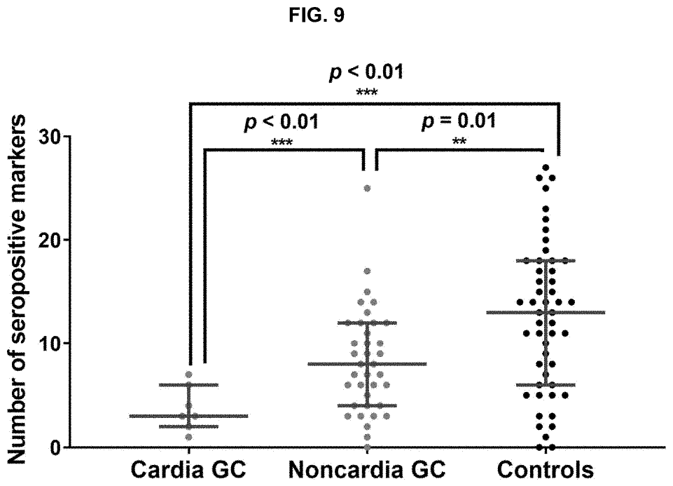

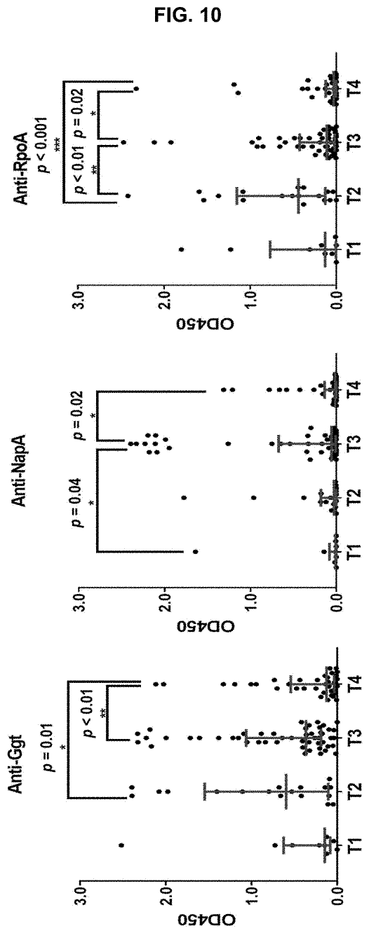

Association of validated antibodies with clinical parameters: Lauren classification and tumor location each showed significant association with validated anti-H. pylori antibodies. Anti-Ggt response were higher in patients with diffuse-type tumors than intestinal-type (p=0.04), and higher in patients with noncardiac GC than cardiac GC (p=0.02) (FIGS. 8A-8B). Overall, patients with cardiac GC had fewer positive antibodies on H. pylori-NAPPA than patients with noncardiac GC, and both cancer groups had fewer antibodies than controls (FIG. 9). Seroreactivity of the three validated antibodies (anti-Ggt, anti-NapA, anti-RpoA) showed significant differences among GC patients with different stages (FIG. 10).

Discussion

H. pylori and humans have coevolved for at least 50,000 years. H. pylori infection is generally acquired early in life and persists life-long, frequently asymptomatically. This implies near-perfect adaptation to its gastric niche and an ability to evade the human immune response. Most H. pylori live superficial to the epithelial cell layer, thus the infection typically does not elicit a strong humoral response. In agreement, our study identified a limited number of immunogenic proteins (3.5%; 53/1527) with >10% seropositivity (in either cases or controls) by screening almost the entire H. pylori proteome. Overall H. pylori seropositivity (combining GC cases and controls) for the evaluated proteins ranged from 0% to 77%. Among all seroreactive antibodies, anti-GroEL best reflected the overall H. pylori serostatus as compared to wcELISA. It was determined that the antibody response to H. pylori was associated with both tumor location and histology. These results also support previous serology studies showing that the H. pylori association with noncardiac is stronger than with cardiac GC, and that the diffuse-type GC is associated with higher anti-Ggt.

Currently, there are no anti-H. pylori antibodies that meet the criteria for clinical utility in GC diagnosis or screening. The present study represents the most comprehensive comparison to date of immunoproteomic profiles between GC cases and controls, coupled with ELISA verification and blinded independent validation. Comparing groups with similarly high overall seroprevalence of H. pylori as determined by wcELISA, inverse associations with GC for anti-Ggt, anti-NapA, anti-PalA, anti-HslU, anti-RpoA, and anti-KatA were found. Proteins targeted by the six validated antibodies may represent novel vaccine candidates that warrant further investigation. Their annotated biological functions include various bacterial life processes, including DNA recombination, transcription, protein synthesis and degradation, and protection from oxidative stress. Notably, these proteins also interact with host cells and modulate both innate and adaptive immune responses. HP0243 (NapA) is a neutrophil-activating protein, which is observed in both the cytosol and bacterial culture medium. It belongs to the well-conserved Dps family and plays dual roles in H. pylori: it recruits neutrophils and monocytes and stimulates reactive oxygen intermediates (ROI) while it protects H. pylori by combating against the ROI. HP1293 (RpoA) is the DNA-directed RNA polymerase subunit alpha which is involved in transcription. HP0516 (HslU) is an ATP-dependent protease ATPase subunit that functions as a proteasome-like degradation complex and has a chaperone-like activity, which is involved in protein unfolding and proteolysis. HP0875 (KatA) is a catalase and protects cells from the toxic effects of hydrogen peroxide. Catalase has been proposed as a target for immunization. HP1118 (Ggt) encodes the gamma-glutamyltranspeptidase, which not only supports initial H. pylori colonization, but also functions as a key virulence factor in inhibition of T-cell proliferation. HP0371 (FabE) is a biotin carboxyl carrier protein of acetyl-CoA carboxylase, which is involved in fatty acid biosynthesis.

Previous data on H. pylori protein subcellular localization are incomplete and covers only a subset of the proteome. There was a significant enrichment of MSPs for the 53 proteins with >10% seropositivity. Notably, the six validated antibodies were predicted to be cytoplasmic proteins, which may explain their eliciting strong immune responses. However, three of these six (Ggt, NapA, and KatA) were previously reported to be in the membrane and outer membrane fractions by LC-MS/MS. Therefore, their subcellular localizations are uncertain.

Inverse associations between serological responses to H. pylori-specific proteins and GC has been previously reported. Pan et al. compared antibodies against six proteins (CagA, VacA, GroEL, UreA, HcpC, and Ggt) in patients with precancerous lesions and those with superficial gastritis, and reported that anti-Ggt decreased as patients progressed to more severe lesions. Shakeri et al. tested a 15-antigen panel and found that anti-GroEL and anti-NapA were inversely associated with GC. Notably, inverse associations with both Ggt and NapA were validated in our study. Gastric carcinogenesis is a multistep process with well-defined histological stages: H. pylori infection of normal gastric mucosa→non-atrophic gastritis→atrophic gastritis→intestinal metaplasia dysplasia→GC. At the stage of atrophy, H. pylori infection is frequently lost due to chronic inflammation that disrupts the stomach mucosa. The lack of bacterial stimulus may explain a reduced humoral response against H. pylori in GC cases. A cumulative effect of antibiotic use may also lead to a decreased antibody response.

Our finding of increased CagA seroreactivity in GC cases compared to controls aligns with previous reports based on prediagnostic and diagnostic samples, as well as studies of preneoplastic lesions. Interestingly, while most H. pylori antibody responses are diminished in GC along with the decrease in H. pylori load, previous studies have shown that anti-CagA antibodies are sustained for a longer time after successful H. pylori eradication. Hypothetically, the increase of anti-CagA antibodies might stem from the CagA-positive strains surviving in GC cases and expressing the corresponding oncoprotein during progression (FIGS. 5A-5B).

Our study has several strengths. First, we used a well-validated and highly reproducible state-of-the-art microarray technology. Secondly, we validated the candidate antibodies by a stringent two-step ELISA that included verification and blinded testing on an independent sample set. Importantly, we tested well-characterized samples with high prevalence of H. pylori infection. The protein set displayed on the H. pylori-NAPPA arrays was based on two reference strains isolated from patients with European ancestry. Although comprehensive, our H. pylori-NAPPA did not include a universal set of proteins, as the panproteome of H. pylori is still being determined.

The six antibodies identified in this study could have a direct application, in combination with other serologic biomarkers, for triage of high-risk individuals in GC screening. Future studies of humoral immunoproteomic profiles should address the various stages of gastric carcinogenesis, and longitudinal samples may inform whether changes in humoral response precede GC development. Decreased antibody response (i.e., decreased seroreactivity) to specific H. pylori proteins may have broader implications as an indicator of disease severity, progression and/or etiologic mechanisms.

TABLE 4
Seropositivity of 15 anti-H. pylori IgA antibodies on NAPPA

| | | | Seropositive (%) | |
|---|---|---|---|---|
| Protein | Names | Full name | Controls | Cases |
| HP1341 | TonB | Energy transducer TonB | 20 | 14 |
| HP0923 | Omp22 | Outer membrane protein (Omp22) | 20 | 10 |
| HP0477 | Omp12 | Outer membrane protein (Omp12) | 18 | 8 |
| HP1172 | GlnH | Glutamine ABC transporter substrate-binding protein | 16 | 16 |
| | | GlnH | | |
| HP0383 | | Hypothetical protein | 14 | 4 |
| jhp_0532 | Nth | Endonuclease III | 12 | 8 |
| HP0185 | | Hypothetical protein | 12 | 8 |
| HP0912 | Omp20 | Outer membrane protein (Omp20) | 10 | 4 |
| HP0685 | FliP | Flagellar biosynthetic protein | 10 | 6 |
| HP0599 | HylB | Methyl-accepting chemotaxis protein | 8 | 12 |
| HP1177 | Omp27 | Outer membrane protein (Omp27) | 6 | 14 |
| HP0845 | ThiM | Hydroxyethylthiazole kinase | 6 | 10 |
| HP0667 | | Mrr_cat_2 domain-containing protein | 4 | 10 |
| HP1244 | RpsR | 30S ribosomal protein S18 | 2 | 10 |
| HP1125 | Omp18 | Peptidoglycan associated lipoprotein | 2 | 10 |

TABLE 5
Relative seroprevalence of 23 anti-H. pylori proteins elevated in controls on NAPPA

| | | | Relative seroprevalence | |
|---|---|---|---|---|
| | | | (%) | |
| Protein | Names | Full name | Controls* | GC cases** |
| HP0371 | AccB, FabE | Biotin carboxyl carrier protein | 36 | 0 |
| HP0153 | RecA | Recombinase RecA | 32 | 2 |
| HP1527 | ComH | Hypothetical protein | 30 | 0 |
| HP0516 | HslU | ATP-dependent protease ATP-binding | 26 | 4 |
| | | subunit HslU | | |
| HP1124 | | Hypothetical protein | 26 | 0 |
| HP1379 | Ion | ATP-dependent protease | 26 | 0 |
| HP0908 | FlgE | Flagellar hook protein FlgE | 22 | 0 |
| HP1238 | AmiF | Formamidase | 22 | 0 |
| HP1293 | RpoA | DNA-directed RNA polymerase subunit | 22 | 4 |
| | | alpha | | |
| HP0645 | Slt | Lytic murein transglycosylase | 20 | 0 |
| HP0779 | AcnB | Bifunctional aconitate hydratase 2/2- | 20 | 0 |
| | | methylisocitrate dehydratase | | |
| HP0795 | Tig | Trigger factor | 20 | 2 |
| HP0875 | KatA | Catalase | 20 | 0 |
| HP1118 | Ggt | Gamma-glutamyltranspeptidase | 20 | 2 |
| HP0243 | NapA | DNA protection during starvation protein | 18 | 0 |
| HP0407 | BisC | Biotin sulfoxide reductase | 18 | 0 |
| HP0561 | FabG | 3-ketoacyl-ACP reductase | 18 | 2 |
| HP1555 | Tfs | Elongation factor Ts | 18 | 2 |
| HP0129 | | Hypothetical protein | 16 | 0 |
| HP0264 | ClpB | Chaperone protein ClpB | 16 | 0 |
| HP0701 | GyrA | DNA gyrase subunit A | 16 | 4 |
| HP0923 | HopK, Omp22 | Membrane protein | 16 | 0 |
| HP1199 | RpI2, Rpl7, | 50S ribosomal protein L7/L12 | 16 | 2 |
| | RpIL | | | |

*Based on MNI cutoff at 95^{th} percentile in cases;
**Based on MNI cutoff at 95^{th} percentile in controls.

TABLE 6
Subcellular locational analysis of the 53 immunodominant H. pylori proteins

| | No. of responsive | | | | | |
|---|---|---|---|---|---|---|
| | proteins in | | No. responsive | | No. of responsive | |
| Subcellular location | all individuals | | proteins in cancer | | proteins in controls | |
| (Total proteins) | (Percentage of total) | | (Percentage of total) | | (Percentage of total) | |
| Extracellular (15) | 3 | (20.0) | 2 | (13.3) | 3 | (20.0) |
| Periplasmic (12) | 3 | (25.0) | 2 | (16.7) | 3 | (25.0) |
| Outer Membrane (24) | 5 | (20.8) | 3 | (12.5) | 5 | (20.8) |
| Cytoplasmic Membrane | 3 | (1.3) | 3 | (1.3) | 3 | (1.3) |
| (226) | | | | | | |
| Cytoplasmic (631) | 26 | (4.1) | 7 | (1.1) | 26 | (4.1) |
| Unknown (619) | 13 | (2.1) | 9 | (1.5) | 12 | (1.9) |
| Total (1527) | 53 | (3.5) | 26 | (1.7) | 52 | (3.4) |

TABLE 7
Relative seroprevalence among 23 discriminatory anti-H. pylori antibodies comparing all
GC cases vs. whole-cell ELISA-positive controls

| | | Discovery set | Validation set | | | | |
|---|---|---|---|---|---|---|---|
| | | % in | % in | % in | Odds | | |
| Protein | Name | controls* | controls* | cases* | ratio | p-value | Validated |
| HP1118 | Ggt | 22 | 35 | 4 | 0.08 | 4.0E−08 | Yes |
| HP0516 | HslU | 34 | 43 | 16 | 0.26 | 3.4E−05 | Yes |
| HP0243 | NapA | 29 | 25 | 7 | 0.22 | 4.3E−04 | Yes |
| HP1293 | RpoA | 27 | 40 | 16 | 0.29 | 1.6E−04 | Yes |
| HP0371 | AccB, FabE | 22 | 24 | 7 | 0.24 | 9.0E−04 | Yes |
| HP0875 | KatA | 27 | 25 | 10 | 0.33 | 4.5E−03 | Yes |
| HP0795 | Tig | 20 | 29 | 13 | 0.36 | 4.7E−03 | Yes |
| HP0153 | RecA | 34 | 31 | 15 | 0.40 | 8.3E−03 | Yes |
| HP0701 | GyrA | 20 | 20 | 9 | 0.40 | 2.7E−02 | Yes |
| HP0561 | FabG | 22 | 21 | 15 | 0.65 | 2.5E−01 | No |
| HP0779 | AcnB | 29 | 19 | 16 | 0.83 | 6.2E−01 | No |
| HP1379 | Ion | 22 | 23 | 17 | 0.70 | 3.1E−01 | No |
| HP 1527 | ComH | 13 | | | | | |
| HP0407 | BisC | 13 | | | | | |
| HP1555 | Tfs | 13 | | | | | |
| HP0923 | HopK, Omp22 | 13 | | | | | |
| HP1199 | RpI2, Rpl7, | 13 | | | | | |
| | RpIL | | | | | | |
| HP0129 | | 13 | | | | | |
| HP0908 | FlgE | 6 | | | | | |
| HP0645 | Sit | 9 | | | | | |
| HP1124 | | 0 | | | | | |
| HP0264 | ClpB | 0 | | | | | |
| HP1238 | AmiF | 0 | | | | | |

*Based on MNI cutoff at 95^{th} percentile in cases (as determined in discovery set).

1. A method for identifying a subject having increased risk of developing gastric cancer, the method comprising:
(a) reacting a biological sample obtained from a subject with a reagent composition that comprises components for detecting in the biological sample the presence of one or more antibodies selected from anti-HP1118/Ggt, anti-HP0516/HslU, anti-HP0243/NapA, anti-HP1293/RpoA, anti-HP0371/FabE, and anti-HP0875/KatA; and
(b) detecting the presence of the antibodies in the sample, wherein reduced seroreactivity relative to a control for one or more of the antibodies is indicative of a 2- to 8-fold increased risk of gastric cancer.

2. The method according to claim 1, wherein the detected antibodies comprise anti-HP1118, anti-HP0516, and anti-HP0243.

3. The method according to claim 1, wherein the detected antibodies comprise anti-HP1118/Ggt, anti-HP0516/HslU, anti-HP0243/NapA, anti-HP1293/RpoA, anti-HP03 71/FabE, and anti-HP0875/KatA.

4. The method according to claim 1, wherein the reagent composition further comprises a component for detecting the presence and level of anti-CagA antibodies in the sample, and wherein increased seroreactivity for anti-CagA antibodies is indicative of an increased risk of gastric cancer relative to a control.

5. The method according to claim 4, wherein the detected antibodies comprise anti-HP1118/Ggt, anti-HP0516/HslU, anti-HP0243/NapA, and anti-CagA.

6. The method according to claim 5, wherein the detected antibodies comprise anti-HP1118/Ggt, anti-HP0516/HslU, anti-HP0243/NapA, anti-HP1293/RpoA, anti-HP0371/FabE, anti-HP0875/KatA, and anti-CagA.

7. The method according to claim 1, further comprising identifying the subject has having an increased risk of developing a diffuse-type gastric cancer tumor if the subject has a higher anti-HP1118/Ggt response relative to an intestinal-type cancer control.

8. The method according to claim 1, further comprising identifying the subject has having an increased risk of developing a noncardiac-type gastric cancer tumor if the subject has a higher anti-HP1118/Ggt response relative to a cardiac-type gastric cancer control.

9. The method according to claim 1, further comprising administering a vaccine-based gastric cancer treatment to the subject if identified as having an increased risk of gastric cancer.

10. The method of claim 1, wherein the biological sample is one or more of a whole blood sample, a serum sample, and a plasma sample.

11. The method of claim 1, wherein the method detects gastric cancer prior to symptom onset.

12. A method to detect gastric cancer comprising:
(a) reacting a biological sample obtained from a subject with a reagent composition that comprises components for determining a level of antibodies to one or more of 53 H. pylori proteins listed in Table 1 are present in the sample;
(b) determining levels of the antibodies in the biological sample; and
(c) comparing the levels to predetermined values indicative of gastric cancer, wherein if the level of antibodies in the biological sample falls within the predetermined values indicative of gastric cancer, the level in the biological sample indicates that the subject has gastric cancer.

13. The method according to claim 12, wherein the antibodies comprise anti-HP1118/Ggt, anti-HP0516/HslU, anti-HP0243/NapA.

14. The method according to claim 13, wherein the detected antibodies comprise anti-HP1118/Ggt, anti-HP0516/HslU, anti-HP0243/NapA, anti-HP1293/RpoA, anti-HP0371/FabE, and anti-HP0875/KatA.

15. The method of claim 12, wherein the reagent composition further comprises a component for determining a level of anti-CagA antibodies in the sample, wherein if the level of anti-CagA antibodies in the biological sample falls within anti-CagA antibody levels of a reference sample obtained from an individual having gastric cancer, the level in the biological sample indicates that the subject has gastric cancer.

16. The method of claim 12, wherein the predetermined values are obtained from a reference sample obtained from an individual or a group of individuals having gastric cancer.

17. The method of claim 12, further comprising administering a gastric cancer treatment to the subject if identified as having gastric cancer.

18. The method of claim 12, wherein the biological sample is one or more of a whole blood sample, a serum sample, and a plasma sample.

19. (canceled)

20. A method of determining an H. pylori antibody signature comprising antibodies, contained in a biological sample from an individual, that specifically bind to immobilized H. pylori antigens, the method comprising:
(a) contacting the sample to a panel of immobilized H. pylori antigens under conditions that promote formation of antigen-antibody complexes; and
(b) identifying complexes formed by immobilized H. pylori antigens and antibody in the sample, to determine an H. pylori antibody signature.

21.-25. (canceled)

26. A kit for determining and/or detecting at least one biomarker associated with gastric cancer, the kit comprising a reagent composition that comprises components for detecting in a biological sample the presence of one or more antibodies selected from anti-HP1118/Ggt, anti-HP0516/HslU, anti-HP0243/NapA, anti-HP1293/RpoA, anti-HP0371/FabE, and anti-HP0875/KatA.

27. (canceled)
